(19) United States
(12) Patent Application Publication
(10) Pub. No.: US 2026/0162548 A1
(43) Pub. Date: Jun. 11, 2026

(54) INFORMATION PROCESSING APPARATUS, INFORMATION PROCESSING METHOD, AND RECORDING MEDIUM

(71) Applicant: NEC Corporation, Tokyo (JP)

(72) Inventors: Terumi Umematsu, Tokyo (JP); Fumiyuki Nihey, Tokyo (JP); Chenhui Huang, Tokyo (JP); Kentaro Nakahara, Tokyo (JP); Kazuyuki Sakurai, Tokyo (JP)

(73) Assignee: NEC Corporation, Tokyo (JP)

(21) Appl. No.: 19/181,583

(22) Filed: Apr. 17, 2025

(30) Foreign Application Priority Data
May 14, 2024 (JP) 2024-078683

(51) Int. Cl. G09B 5/02; A61B 5/00; A61B 5/11; A61B 5/16; G06T 7/00; G16H 20/30; G16H 20/60; G16H 40/67

(52) U.S. Cl. CPC G09B 5/02; A61B 5/112; A61B 5/165; A61B 5/4842; A61B 5/6807; A61B 5/742; G06T 7/0012; G16H 20/30; G16H 20/60; G16H 40/67; G06T 2207/20081; G06T 2207/20084; G06T 2207/30201

(57) ABSTRACT

An information processing device acquires each facial image of a subject by an imaging device for capturing a body of the subject. The information processing device estimates a physical frailty degree of the subject based on each facial image. The information processing device estimates a psychological frailty degree of the subject based on the output of the detection device. The information processing device proposes a method of proposing a method for preventing frailty based on the physical frailty degree and the psychological frailty degree of the subject.

### TECHNICAL FIELD

This disclosure relates to a technique for maintaining the health of a subject.

### BACKGROUND ART

‘Frail’ is a Japanese translation of ‘frailty’ and is a concept proposed by the Japan Geriatrics Society in 2014, hereinafter referred to as ‘frailty’. Specifically, the frailty is a state that lies between being healthy and requiring nursing care, characterized by a decline in physical and cognitive functions. The frailty consists of a physical factor such as muscle weakness, mental and a psychological factor such as dementia and depression, and social a factor such as living alone or financial difficulties.

Even if an individual is in a frailty state, appropriate treatment and prevention measures can greatly reduce a likelihood of progressing to a state requiring the nursing care. Therefore, it is important to maintain at least one of the physical, psychological, or social factors in a healthy state to prevent frailty.

Traditionally, a frailty prevention involved assessing a condition of the individual through questionnaires and recommending actions such as proper nutrition, exercise, and social participation based on the results. Additionally, Patent Document 1 describes a health behavior recommendation system that encourages health-promoting actions to prevent the frailty based on voice and movements of the individual.
Patent Document 1: International Publication No. WO 2022/224621

### SUMMARY

One object of the present disclosure is to provide an appropriate proposal for preventing frailty based on information regarding a state of a subject.

According to an example aspect of the present invention, there is provided an information processing device comprising:
at least one memory configured to store instructions; and
at least one processor configured to execute the instructions to:
acquire each facial image of a subject by an imaging device for capturing a body of the subject;
estimate a physical frailty degree of the subject based on each facial image;
estimate a psychological frailty degree of the subject based on the output of a detection device different from the imaging device; and
propose a method for preventing frailty by displaying, on a user interface, a personalized recommendation of at least one dietary plan and exercise regime, based on the physical frailty degree and the psychological frailty degree of the subject.

According to another example aspect of the present invention, there is provided an information processing device including a detection device for detecting a state of a body of a subject, the information processing method comprising:
acquiring each facial image of a subject by an imaging device for capturing a body of the subject;
estimating a physical frailty degree of the subject based on each facial image;
estimating a psychological frailty degree of the subject based on the output of a detection device different from the imaging device; and
proposing a method for preventing frailty by displaying, on a user interface, a personalized recommendation of at least one dietary plan and exercise regime, based on the physical frailty degree and the psychological frailty degree of the subject.

According to still another example aspect of the present invention, there is provided a non-transitory computer-readable recording medium storing a program causing a computer including a detection device for detecting a state of a body of a subject to execute processing of:
acquiring each facial image of a subject by an imaging device for capturing a body of the subject;
estimating a physical frailty degree of the subject based on each facial image;
estimating a psychological frailty degree of the subject based on the output of a detection device different from the imaging device; and
proposing a method for preventing frailty by displaying, on a user interface, a personalized recommendation of at least one dietary plan and exercise regime, based on the physical frailty degree and the psychological frailty degree of the subject. EFFECT

According to the present disclosure, it is possible to make an appropriate proposal for preventing frailty based on information regarding a state of a subject.

### BRIEF DESCRIPTION OF THE DRAWINGS

FIG. 1 illustrates an example of a schematic configuration of a frailty prevention system.

FIG. 2A is a block diagram illustrating an example of a hardware configuration of a server and FIG. 2B is a block diagram illustrating an example of a hardware configuration of a user terminal.

FIG. 3 is a block diagram illustrating an example of a functional configuration of the server.

FIG. 4A and FIG. 4B illustrate examples of a proposal for a frailty prevention method.

FIG. 5 is a flowchart of a frailty prevention.

### EXAMPLE EMBODIMENTS

Preferred example embodiments of the present disclosure will be described with reference to the accompanying drawings.

### EXAMPLE EMBODIMENT

### (Configurations)

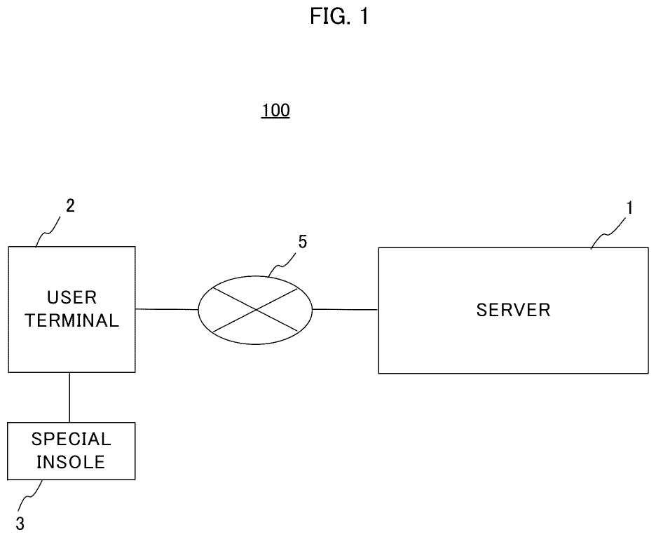

FIG. 1 is an example of a schematic configuration of a frailty prevention system 100 to which an information processing device of the present disclosure is applied. The frailty prevention system 100 is a system that can make an appropriate proposal to prevent frailty based on information concerning a state of a subject.

Here, the frailty falls between a healthy state and a state of requiring the nursing care, shows a decline in physical and cognitive functions, and consists of a physical frailty such as muscle weakness, a psychological frailty such as dementia and depression, and a social frailty such as living alone and economic deprivation.

In the frailty prevention system 100, a server 1 and a user terminal 2 are connected to each other so as to be capable communicating with each other via a network 5 such as the Internet. The user terminal 2 may be a smart phone, a tablet, a PC, or a similar device used by a user who is a subject of concern for frailty (hereinafter also referred to as a “subject”) to capture each facial image of the subject and transmits one or more facial images to the server 1. For instance, the subject may be an elderly person who is anxious concerning a health condition or a social position. Also, the user terminal 2 is connected to the special insole 3 mounted by the subject, communicates via a short-range wireless communication, a predetermined network or the like, acquires walking data of the subject from the special insole 3, and transmits the walking data to the server 1.

The server 1 is an information processing device that processes, stores, transmits, and receives various data, and estimates a psychological frailty degree and a physical frailty degree of the subject from the one or more facial images and the walking data of the subject. Moreover, the server 1 proposes a frailty prevention method based on the estimated psychological frailty degree and the estimated physical frailty degree. The frailty prevention method refers to appropriate activities for maintaining health, such as diet, exercise, and social participation, tailored to the state of each subject. For example, the frailty prevention method includes at least one dietary plan and exercise regime. The dietary plan includes parameters such as nutrients to be ingested, foods, amounts, and cooking methods. The exercise regime includes parameters such as exercise type, intensity, frequency, and duration.

Note that, in the present disclosure, the server 1 acquires the one or more facial images of the subject from the user terminal 2 via the network 5, but the present disclosure is not limited thereto, and for instance, the one or more facial images may be acquired using an external storage such as a USB (Universal Serial Bus) memory without using the network 5, and a method by which the server 1 acquires the one or more facial images can be set arbitrarily. In addition, it is preferable that the one or more facial images are to be of the subject in a conversational state. At this time, a person conversing with the subject is not limited to a medical professional, but may be, for instance, a family member or the like.

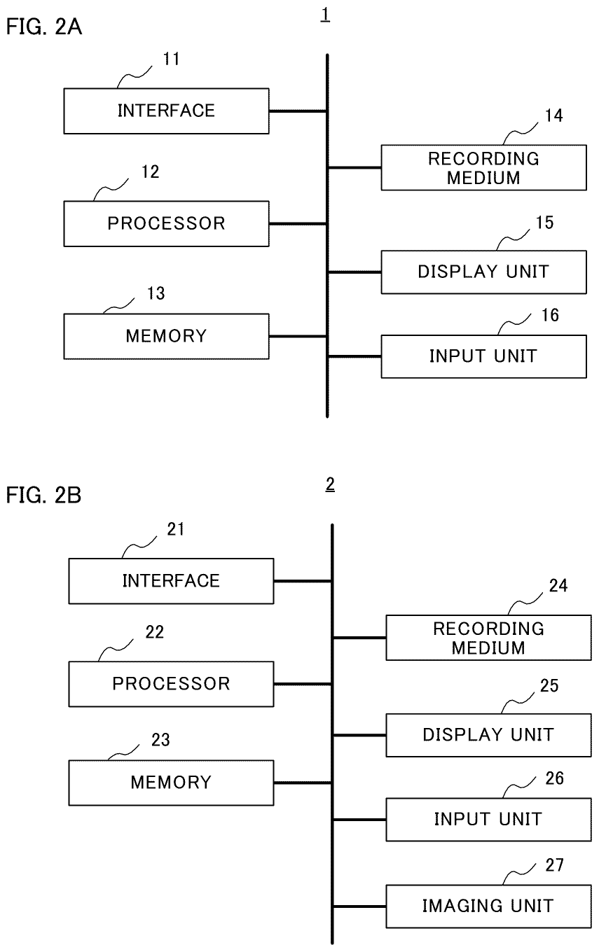

FIG. 2A is a block diagram illustrating an example of a hardware configuration of the server 1. As shown in FIG. 2A, the server 1 includes an interface (Interface) 11, a processor 12, a memory 13, a recording medium 14, a display unit 15, and an input unit 16.

The interface 11 exchanges data with the user terminal 2. The interface 11 is used to receive each facial image and the walking data of the subject from the user terminal 2. Also, the interface 11 is used by the server 1 to exchange data with a predetermined device connected via wired or wireless communications.

The processor 12 is a computer such as a CPU (Central Processing Unit) and controls the entire server 1 by executing programs prepared in advance. Incidentally, as the processor 12, a CPU, a GPU (Graphics Processing Unit), a DSP (Digital Signal Processor), a MPU (Micro Processing Unit), a FPU (Floating Point number Processing Unit), a PPU (Physics Processing Unit), a TPU (Tensor Processing Unit), a quantum processor, a microcontroller, or a combination thereof can be used.

The memory 13 is formed by a ROM (Read Only Memory) and a RAM (Random Access Memory). The memory 13 stores programs executed by the processor 12. The memory 13 is also used as a working memory during various processes performed by the processor 12.

The recording medium 14 is a non-volatile and non-transitory recording medium such as a disk-shaped recording medium or a semiconductor memory, and is formed to be detachable from the server 1. The recording medium 14 records various programs executed by the processor 12. If the server 1 executes the frailty prevention process, programs recorded on the recording medium 14 are loaded into the memory 13 and executed by the processor 12.

The display unit 15 is, for instance, an LCD (Liquid Crystal Display), and displays a predetermined image. The input unit 16 is a keyboard, a mouse, a touch panel, or the like, and is used by an operator who manages the server 1.

FIG. 2B is a block diagram illustrating an example of a hardware configuration of the user terminal 2. As illustrated in FIG. 2B, the user terminal 2 includes an interface 21, a processor 22, a memory 23, a recording medium 24, a display unit 25, an input unit 26, and an imaging unit 27.

The interface 21 exchanges data with the server 1 through the network 5. The interface 21 is used to transmit each facial image and the walking data of the subject to the server 1, or to receive the appropriate proposal according to one or more frailty degrees of the subject from the server 1.

The processor 22 is a computer, such as a CPU, and controls the entire user terminal 2 by executing a program prepared in advance. As the processor 22, a CPU, a GPU, a DSP, a MPU, a FPU, a PPU, a TPU, a quantum processor, a microcontroller, or a combination thereof may be used.

The memory 23 is formed by a ROM, a RAM or the like. The memory 23 stores programs executed by the processor 22. Also, the memory 23 is used as a working memory during various process operations by the processor 22.

The recording medium 24 is a non-volatile and non-transitory recording medium such as a disk-shaped recording medium or a semiconductor memory and is detachably formed to the user terminal 2. The recording medium 24 records various programs executed by the processor 22. The display unit 25 is, for instance, an LCD, and displays a predetermined image. The input unit 26 is a touch panel or the like and is used if the user performs a predetermined operation. The imaging unit 27 includes a camera, and acquires still image data or moving image data being captured.

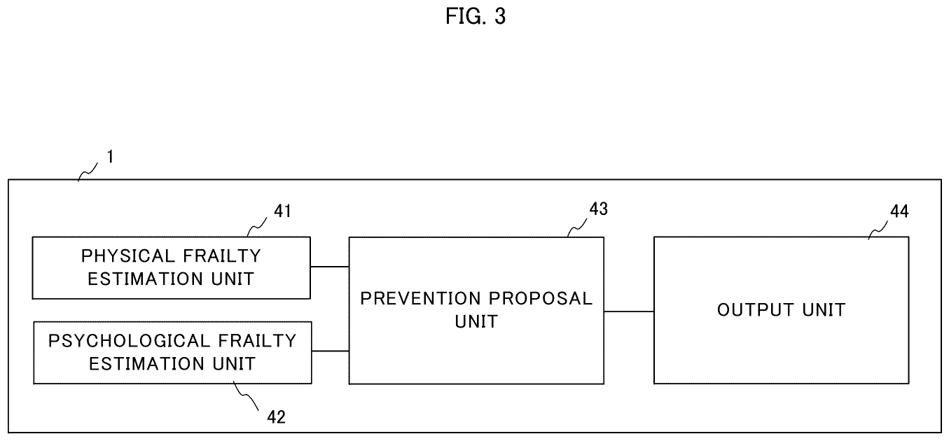

FIG. 3 is a block diagram illustrating an example of a functional configuration of the server 1. The server 1 functionally includes a physical frailty estimation unit 41, a psychological frailty estimation unit 42, a prevention proposal unit 43, and an output unit 44. The physical frailty estimation unit 41, the psychological frailty estimation unit 42, the prevention proposal unit 43, and the output unit 44 are realized by the processor 12 executing corresponding programs.

The physical frailty estimation unit 41 estimates the physical frailty degree of the subject. The physical frailty is a condition in which low nutrition and muscle weakness reduce a distance traveled and a function of movement. The physical frailty degree is a degree of functional decline in the distance traveled and movement, and is expressed as one of three risk levels: 0, 1, and 2, in the present example embodiment. For instance, the risk level 0 is a healthy condition without the physical frailty. The higher the risk level, the greater the physical frailty degree, with the risk level 1 indicating a state just short of frailty, known as pre-frail, and the risk level 2 signifying a state just short of requiring nursing care due to physical functional impairment.

Specifically, the special insole 3 is an insole to be used by wearing it on a shoe of the subject, and the sensor is embedded. The sensor consists of a IMU (Inertial Measurement Unit), a chronic measuring unit, a MPU (Micro Processor Unit), a BLE (Bluetooth Low Energy) module, and a battery.

The user terminal 2 stores a dedicated application corresponding to the special insole 3 in an executable state. The subject registers physical information such as gender, date of birth, height, and weight on the dedicated application by a predetermined operation using the user terminal 2. Moreover, the dedicated application acquires the walking data measured by the sensor from the special insole 3 at a predetermined timing, and transmits the walking data together with the physical information of the subject to the server 1.

The physical frailty estimation unit 41 calculates walking features such as walking speed or stride length of the subject and physical ability estimate values such as lower limb muscle strength and balance ability, by analyzing the walking data and physical information acquired from the user terminal 2. Next, the physical frailty estimation unit 41 estimates a sickness risk score on the basis of the physical information, the calculated walking features, and the physical ability estimate values. Subsequently, the physical frailty estimation unit 41 estimates the physical frailty degree of the subject by normalizing the estimated sickness risk score and classifying the estimated physical frailty degree into one of three risk levels from 0 to 2.

Note that a technique for estimating the physical frailty degree based on the walking data detected by the special insole 3 worn by the subject is described in, for instance, a “PCT/JP2023/023043”, filed by the same applicant as this application, and its specification is incorporated herein by reference.

The psychological frailty estimation unit 42 estimates the psychological frailty degree of the subject. The psychological frailty is a condition in which cognitive function declines due to aging, depression, etc. The psychological frailty degree is a degree of cognitive decline, and is expressed as one of three values: 0, 1, or 2, in the present example embodiment. For instance, the risk level 0 is a healthy condition without the psychological frail. The higher the risk level, the greater the psychological frailty degree.

Concretely, the user terminal 2 acquires each facial image of the subject during the conversation, and transmits one or more facial images to the server 1. In a case where the subject in the one or more facial images displays wakefulness and a video is of a predetermined duration, the psychological frailty estimation unit 42 estimates the cognitive function of the subject based on the one or more facial images. The psychological frailty estimation unit 42 calculates features related to a degree of eye opening for eyes of the subject from a facial video, estimates the cognitive function based on a rate of eyes closed and an eyelid movement speed which are acquired from the features, outputs “cognitively healthy”, “mild cognitive impairment”, or “dementia” as an estimation result. The psychological frailty estimation unit 42 estimates, as the estimation result, the psychological frailty degree as the risk level 0 if “cognitively healthy” is output, as the risk level 1 if “mild cognitive impairment” is output, and as the risk level 2 if “dementia” is output.

The psychological frailty degree estimation unit 42 may output a value, as the estimation result, not limited to “cognitive healthy,” “mild cognitive impairment,” or “dementia,” but equivalent to a score of the Mini-Mental State Examination (MMSE) which is one of cognitive function evaluations. In this case, the psychological frailty degree estimation unit 42 may estimate the psychological frailty degree of the subject by normalizing the output value equivalent to the score of the MMSE and classifying the normalized value into one of the three risk levels: 0, 1, or 2.

It should be noted that, based on the facial image of the subject, a technique for estimating the psychological frailty degree is described in, for instance, PCT/JP2023/041209, filed by the same applicant as this application, and its specification is incorporated herein by reference.

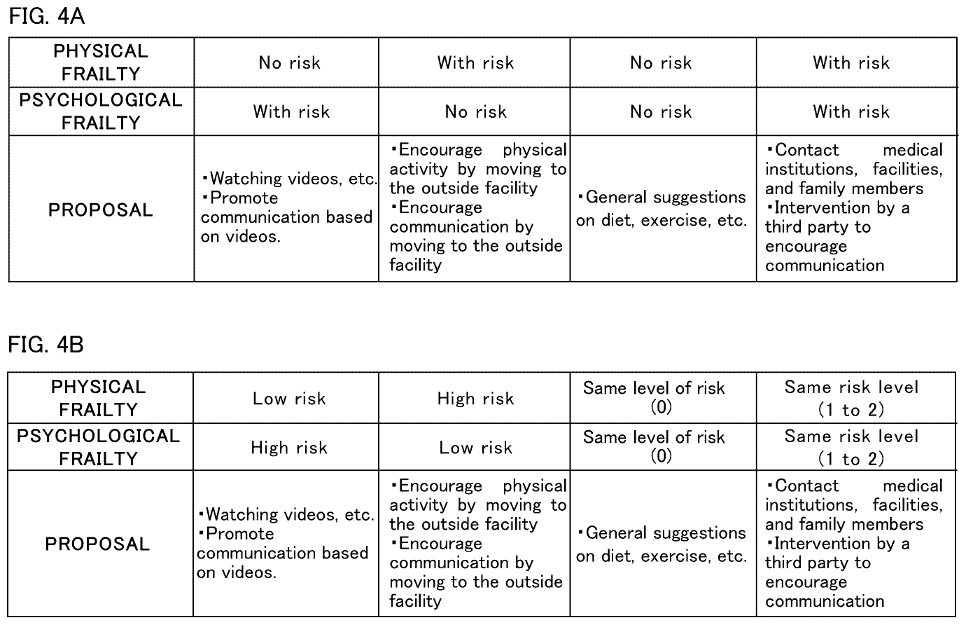

The prevention proposal unit 43 proposes an optimized frailty prevention method for the subject based on the physical frailty degree and the psychological frailty degree of the subject. FIG. 4A and FIG. 4B illustrates examples of a proposal for a frailty prevention method.

Specifically, in a first method of proposing the frailty prevention method, the prevention proposal unit 43 first determines whether risks of the physical frailty and the psychological frailty of the subject is equal to or greater than respective threshold values. In the present example embodiment, the threshold values are set to the “risk level 1”. Therefore, the prevention proposal unit 43 determines that the subject has a risk of a physical frailty if the physical frailty degree indicates the risk level 1 or 2, and has no risk of the physical frailty if the physical frailty degree indicates the risk level 0. In addition, the prevention proposal unit 43 determines that the subject has a risk of psychological frailty if the psychological frailty degree indicates the risk level 1 or 2, and has no risk of the psychological frailty if the psychological frailty degree indicates the risk level 0.

Next, the prevention proposal unit 43 proposes the frailty prevention method as shown in FIG. 4A according to whether there is a risk of physical frailty and whether there is a risk of psychological frailty. For instance, in a case where the subject has no risk of physical frailty but has only the risk of phycological frailty, the prevention proposal unit 43 proposes to watch a video or the like for mood enhancement. Moreover, the prevention proposal unit 43 proposes promotion of communication based on the video. Thus, providing opportunities for communication and encouraging social frailty prevention, such as preventing social isolation and solitary eating, it is possible to promote improvement of a quality of life (QOL) of the subject. Accordingly, it is possible to provide overall flail prevention. In addition, exercising for the physical frailty prevention and watching videos for the psychological frailty prevention necessitate self-motivation of the subject, and it is often difficult for the subject to start and maintain these activities. On the other hand, the social frailty prevention which enhances communications of the subject and prevents the social isolation is effective because third-party intervention is feasible, regardless of a voluntary action of the subject.

In a case where the subject has only the risk of the physical frailty and has no risk of the psychological frailty, the prevention proposal unit 43 proposes exercise by moving to an external facility in order to increase muscular strength. In addition, the prevention proposal unit 43 proposes to promote communications by moving to an external facility.

In a case where the subject presents no risk to both the physical frailty and the psychological frailty, the prevention proposal unit 43 proposes a general health maintenance through diet and exercise. In contrast, in a case where both the physical frailty and the psychological frailty are at risk, the subject has a high risk of requiring nursing care, and the prevention proposal unit 43 proposes contacting medical institutions, facilities, and family members. In addition, the prevention proposal unit 43 proposes the promotion of communication through third-party intervention. Here, the third-party intervention includes, but is not limited to, human communication, and may also encompass conversations with voice-recognizable interactive AI (Artificial Intelligence).

Moreover, in another method of proposing the frailty prevention method, the prevention proposal unit 43 first determines which of the physical frailty and the psychological frailty poses high risk for the subject. In the present example embodiment, respective numerical values indicating risk levels of the physical frailty degree and the psychological frailty degree are compared with each other, and a higher value is determined to represent a higher frailty risk.

Next, the prevention proposal unit 43 proposes the frailty prevention method, as shown in FIG. 4B, based on whether the subject is at a high risk of physical frailty or psychological frailty. For instance, in a case where the subject is at a higher risk of psychological frailty than of physical frailty, the prevention proposal unit 43 proposes watching videos and similar activities for mood enhancement. In addition, the prevention proposal unit 43 proposes the promotion of communication based on the video. Thus, the prevention proposal unit 43 indicates which of physical and psychological frailty is at higher risk and, and proposes a method to reduce the social frailty.

In a case where the physical frailty degree is higher than the psychological frailty degree, the prevention proposal unit 43 proposes exercise by moving to the external facility in order to increase muscular strength. In addition, the prevention proposal unit 43 proposes to promote communications by moving to the external facility.

In a case where both the physical frailty degree and the psychological frailty degree are at risk level 0, due to each indicating low risk, the prevention proposal unit 43 proposes the general health maintenance through diet and exercise. In contrast, in a case where each of the physical frailty degree and the psychological frailty degree indicates the risk level 1 or 2, since the subject is at a high risk of requiring nursing care, and the prevention proposal unit 43 proposes contacting medical institutions, facilities, and family members. In addition, the prevention proposal unit 43 proposes promotion of communication through the third-party intervention.

Incidentally, the present disclosure is not limited to the proposal shown in FIG. 4A and FIG. 4B, and for instance, it is possible to propose an effective diet as a method of the physical frailty prevention, or propose a cognitive function test as a method of the psychological frailty prevention. In other words, contents of the proposal can be set arbitrarily.

In addition, since the social frailty can be estimated by an amount of conversation of the subject, the prevention proposal unit 43 may determine the risk by estimating the social frailty degree of the subject in advance based on the amount of conversation with the interactive AI, and propose the promotion of communication in accordance with a determination result.

The output unit 44 outputs the proposal generated by the prevention proposal unit 43. Specifically, the output unit 44 transmits the proposal generated by the prevention proposal unit 43 as data to the user terminal 2 for display or audio output. Thus, based on the physical and psychological frailty degree of the subject, it is possible to propose an appropriate frailty prevention method according to an individual situation of each subject.

Furthermore, in the above-described configuration, the physical frailty estimation unit 41, the psychological frailty estimation unit 42, and the prevention proposal unit 43 of the server 1 are respective examples of the physical frailty estimation unit, the psychological frailty estimation unit, and the prevention proposal unit according to the present disclosure.

### (Frailty Prevention Process)

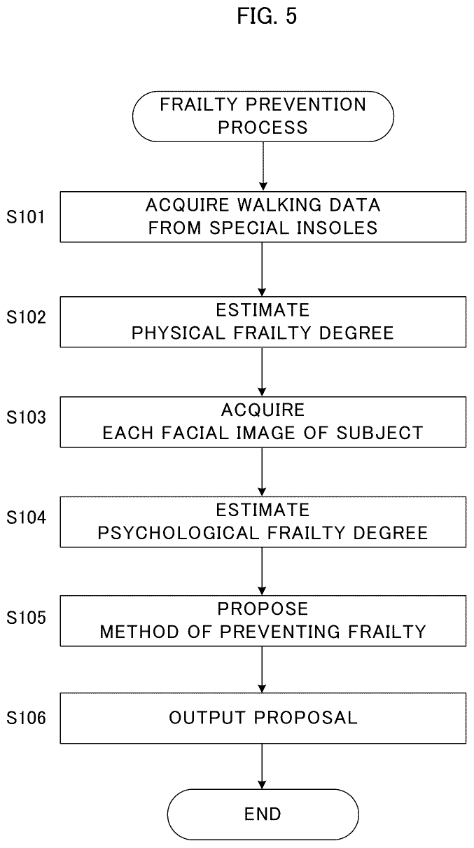

Next, a frailty prevention process performed by the server 1 will be described. FIG. 5 is a flowchart of the frailty prevention process performed by the server 1. This process is realized by executing a program prepared in advance by the processor 12 shown in FIG. 2A.

First, the server 1 acquires the walking data of the subject from the special insole 3 through the user terminal 2 (step S101). Next, the server 1 estimates the physical frailty degree of the subject based on the walking data (step S102). Moreover, the server 1 acquires, from the user terminal 2, one or more facial images obtained by capturing each facial image of the subject during conversations (step S103). Next, the server 1 estimates the psychological frailty degree of the subject based on the acquired one or more facial images (step S104).

The server 1 proposes an optimized frailty prevention method based on the physical frailty degree and the psychological frailty degree of the subject (step S105). Next, the server 1 outputs the proposed frailty prevention method (step S106). Specifically, the server 1 transmits the proposed frailty prevention method to the user terminal 2 as data, and notifies an appropriate frailty prevention method according to the situation of the subject by display or audio output. After that, the server 1 terminates the frailty prevention process.

According to the frailty prevention system 100, it is possible to notify the appropriate frailty prevention method according to the individual situation of each subject by using the data that can be obtained on a daily basis without imposing a load on the subject. Therefore, it is possible to maintain the health of the subject and prevent the frailty.

In the present example embodiment, the frailty prevention method proposed by the server 1 is output to the user terminal 2 to notify the subject; however, the present disclosure is not limited thereto, and the physical frailty degree or the psychological frailty degree may be output in conjunction with the frailty prevention method to notify the subject. In this case, the server 1 may notify one of or both the physical frailty degree and the psychological frailty degree at the risk level or may notify presence or absence of the risk for each thereof.

Furthermore, in the present example embodiment, the frailty prevention method proposed to the subject is output to the user terminal 2 used by the subject; however, the present disclosure is not limited thereto, and for instance, IDs and mail addresses of terminal devices, which are used by family members of the subject, facilities of the subject such as a nursing home in which the subject is resident, a regular medical facility of the subject, and the like, may be stored in advance in the server 1, and the frailty prevention method is output to these terminals in order to notify the frailty prevention method to relevant parties of the subject. At this time, the server 1 may output any one or more of the frailty prevention method, the physical frailty degree, and the psychological frailty degree of the subject to these terminals to notify the relevant parties of the subject.

### First Modification

In the present example embodiment, the physical frailty estimation unit 41 estimates the physical frailty degree based on the walking data acquired from the special insole 3; however, the present disclosure is not limited thereto, and a physical frailty state may be estimated based on the data detected using a detection device that detects a state of a body of the subject. The detection device may be a camera mounted on a smartphone or the like capable of capturing the body of the subject. For instance, the physical frailty estimation unit 41 may estimate the physical frailty degree based on data such as a step count and a heart rate acquired from a pedometer, a smart watch worn by the subject or a sensor worn on the body of the subject. In addition, it is possible to acquire data of grip strength which tends to be proportional to an amount of muscle in the whole body, and estimate the physical frailty degree based on the grip strength.

The physical frailty estimation unit 41 may estimate the physical frailty degree based on the features of the subject. In this case, the physical frailty estimation unit 41 extracts the features of the face based on the one or more facial images, and detects movement of the muscle of the face by analyzing the features. The physical frailty estimation unit 41 estimates the physical frailty degree based on the movement of the muscle of the face.

In addition, the physical frailty estimation unit 41 may estimate the physical frailty degree more precisely by combining a plurality of methods such as one or more facial images and the walking data.

In addition, although the physical frailty degree is represented by one of three risk levels 0 to 2, the present disclosure is not limited thereto, and levels of the risk can be arbitrarily set. In addition, instead of such a level classification, for instance, a disease risk score, a numerical value of the grip strength, or similar metrics may be used to represent the frailty degree.

### Second Modification

In the present example embodiment, the psychological frailty estimation unit 42 estimates the psychological frailty degree based on facial images forming the video, but the present disclosure is not limited thereto, and the psychological frailty degree may be estimated based on a plurality of consecutive still images capturing the subject.

Moreover, the psychological frailty estimation unit 42 is not limited to the facial image, and may estimate the psychological frailty degree based on, for instance, the walking data acquired from the special insole 3 or a score of a spatial reasoning test of a neuropsychological test. In a case of estimating based on the walking data, by analyzing the walking data of the subject, the psychological frailty estimation unit 42 detects asymmetries between left and right sides, such as a frequency of foot collisions or a tendency to bump a little toe on one side more often, and estimates a spatial reasoning ability. Since the cognitive function tends to be proportional to the spatial reasoning ability, the psychological frailty estimation unit 42 estimates the psychological frailty degree based on the spatial reasoning ability and the score of the spatial reasoning test which are estimated based on the walking data.

Moreover, the psychological frailty estimation unit 42 may estimate the psychological frailty degree more precisely by combining a plurality of methods such as one or more facial images and the walking data.

Furthermore, although the physical frailty degree is represented by one of three risk levels 0 to 2, the present disclosure is not limited thereto, and levels of the risk can be arbitrarily set. Alternately, instead of such a level classification, for instance, a disease risk score, a numerical value of the grip strength, or similar metrics may be used to represent the frailty degree.

### Third Modification

The physical frailty degree may be estimated using a physical frailty estimation model that is a machine learning model. For instance, the physical frailty estimation unit 41 may construct the physical frailty estimation model that outputs an optimized physical frailty degree in response to an input of the walking data. For the construction (generation) of the physical frailty estimation model, training data are used. The training data are data in which input data to be entered in training of the physical frailty estimation model is associated with correct answer data corresponding to the input data. The input data are various walking data, and the correct answer data indicate the physical frailty degree. The physical frailty estimation unit 41 trains the physical frailty estimation model to output the physical frailty degree based on the walking data input as the input data. For instance, a model using a neural network or the like are exemplified as a method of machine learning. According to this, the physical frailty estimation unit 41 can use the physical frailty degree output by the physical frailty model as an estimation result.

### Fourth Modification

The psychological frailty degree may be estimated using a psychological frailty estimation model which is a machine learning model. For instance, the psychological frailty estimator 42 may construct the psychological frailty estimation model that outputs an optimized psychological frailty degree in response to an input of each facial image. For the construction (generation) of the psychological frailty estimation model, the training data are used. The training data are data in which the input data to be entered in training of the psychological frailty estimation model is associated with the correct answer data corresponding to the input data. The input data correspond to various facial images, and the correct answer data indicate the psychological frailty degree. The psychological frailty estimation unit 42 trains the psychological frailty estimation model to output the psychological frailty degree based on the facial images input as the input data. For instance, a model using a neural network or the like can be exemplified as a method of machine learning. Therefore, the psychological frailty estimation unit 42 can use the psychological frailty degree output by the psychological frailty model as the estimation result.

### Fifth Modification

In the example embodiment described above, the subject uses the user terminal 2, but the present disclosure is not limited thereto, and the subject may use a user terminal having functions of the server 1. In this case, the user terminal can perform the frailty prevention process performed by the server 1, in order to estimate the physical frailty and the psychological frailty of the subject and to propose the frailty prevention and output that proposal. In other words, the user terminal alone can perform all functions of estimating the physical frailty and the psychological frailty, proposing the frailty prevention and outputting that proposal.

Additionally, some or all of the above embodiments (including modifications, the same applies hereinafter) may also be described as in the following supplementary notes, but are not limited thereto.

### Supplementary Note 1

An information processing device comprising:
a detection device configured to detect a state of a body of a subject;
a physical frailty estimation means configured to estimate a physical frailty degree of the subject based on an output of the detection device;
a psychological frailty estimation means configured to estimate a psychological frailty degree of the subject based on the output of the detection device; and
a prevention proposal means configured to propose a method of proposing a method for preventing frailty based on the physical frailty degree and the psychological frailty degree of the subject.

### Supplementary Note 2

The information processing device according to supplementary note 1, wherein the detection device is a sensor to be worn on the body of the subject.

### Supplementary Note 3

The information processing device according to supplementary note 2, wherein
the detection device is an insole sensor and outputs walking data concerning a walking pattern of the subject; and
the physical frailty estimation means estimates the physical frailty degree of the subject based on the walking data.

### Supplementary Note 4

The information processing device according to supplementary note 1, wherein
the detection device is an imaging device for capturing the body of the subject, and outputs each image of the body of the subject, and
the psychological frailty estimation means estimates the psychological frailty degree of the subject based on each facial image capturing a face of the subject.

### Supplementary Note 5

The information processing device according to supplementary note 1, wherein the method for preventing the frailty is a method of preventing social frailty.

### Supplementary Note 6

The information processing device according to supplementary note 1, wherein
the prevention proposal means determines whether or not the physical frailty degree and the psychological frailty degree of the subject are equal to or greater than respective threshold values, and
the method for preventing the frailty is to reduce the physical frailty which has the physical frailty degree equal to or greater than a first threshold value and is determined to pose high risk, the psychological frailty which has the psychological frailty degree equal to or greater than a second threshold value and is determined to pose high risk, and the social frailty.

### Supplementary Note 7

The information processing device according to supplementary note 1, wherein
the prevention proposal means compares the physical frailty degree with the psychological frailty degree, and determines which one of the physical frailty and the psychological frailty is at higher risk. and
the method for preventing the frailty is to reduce whichever of the physical frailty and the psychological frailty is at the higher risk, and reduce the social frailty.

### Supplementary Note 8

The information processing device according to supplementary note 3, wherein the psychological frailty estimation means estimates the psychological frailty degree of the subject by using a machine learning model which is optimized and trained to output the psychological frailty degree of the subject.

### Supplementary Note 9

An information processing method performed by an information processing device including a detection device for detecting a state of a body of a subject, the information processing method comprising:
estimating a physical frailty degree of the subject based on an output of the detection device;
estimating a psychological frailty degree of the subject based on the output of the detection device; and
proposing a method of proposing a method for preventing frailty based on the physical frailty degree and the psychological frailty degree of the subject.

### Supplementary Note 10

A program causing a computer including a detection device for detecting a state of a body of a subject to execute processing of:
estimating a physical frailty degree of the subject based on an output of the detection device;
estimating a psychological frailty degree of the subject based on the output of the detection device; and
proposing a method of proposing a method for preventing frailty based on the physical frailty degree and the psychological frailty degree of the subject.

### Supplementary Note 11

The information processing device according to supplementary note 4, wherein the psychological frailty estimation means estimates the psychological frailty degree of the subject by using a machine learning model which is optimized and trained to output the psychological frailty degree, in response to an input of one or more facial images of the subject.

### Supplementary Note 12

The information processing device according to supplementary note 3, wherein the psychological frailty estimation means estimates the psychological frailty degree of the subject by estimating a spatial reasoning ability in reference to asymmetries between left and right sides in a frequency of foot collisions based on the walking data.

### Supplementary Note 13

The information processing device according to supplementary note 4, wherein the physical frailty estimation means estimates the physical frailty degree of the subject by detecting a facial muscle movement based on one or more facial images of the subject.

### Supplementary Note 14

The information processing device according to supplementary note 3, wherein
the detection device is an imaging device for capturing the body of the subject and outputs each image of the body of the subject,
the physical frailty estimation means estimates the physical frailty estimation degree based on the walking data of the subject and one or more facial images capturing a face of the subject, and
the psychological frailty estimation means estimates the psychological frailty degree based on the walking data and the one or more facial images.

### Supplementary Note 15

The information processing device according to supplementary note 6, wherein the prevention proposal means
proposes one or more of exercise and diet in a case where the physical frailty poses high risk,
proposes one or more of watching a video and taking a cognitive function test in a case where the psychological frailty poses high risk, and
proposes contacting one or more of a medical institution, a facility, and a family member in a case where both the physical frailty and the psychological frailty pose high risk.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

This application is based upon and claims the benefit of priority from Japanese Patent Application 2024-078683, filed on May 14, 2024, the disclosure of which is incorporated herein in its entirety by reference.

### DESCRIPTION OF SYMBOLS

1 Server
2 User terminal
11, 21 Interface
12, 22 Processor
13, 23 Memory
14, 24 Recording medium
15, 25 Display unit
16, 26 Input unit
27 Imaging unit
41 Physical frailty estimation unit
42 Psychological frailty estimation unit
43 Prevention proposal unit
44 Output unit
100 Frailty prevention system

1. An information processing device comprising:
at least one memory configured to store instructions; and
at least one processor configured to execute the instructions to:
acquire each facial image of a subject by an imaging device for capturing a body of the subject;
estimate a physical frailty degree of the subject based on each facial image;
estimate a psychological frailty degree of the subject based on the output of a detection device different from the imaging device; and
propose a method for preventing frailty by displaying, on a user interface, a personalized recommendation of at least one dietary plan and exercise regime, based on the physical frailty degree and the psychological frailty degree of the subject.

2. The information processing device according to claim 1, wherein the detection device is a sensor to be worn on the body of the subject.

3. The information processing device according to claim 2, wherein
the detection device is an insole sensor and outputs walking data concerning a walking pattern of the subject; and
the processor estimates the physical frailty degree of the subject based on the walking data.

4. The information processing device according to claim 1, wherein the method for preventing the frailty is a method of preventing social frailty.

5. The information processing device according to claim 1, wherein
the processor determines whether or not the physical frailty degree and the psychological frailty degree of the subject are equal to or greater than respective threshold values, and
the method for preventing the frailty is to reduce the physical frailty which has the physical frailty degree equal to or greater than a first threshold value and is determined to pose high risk, the psychological frailty which has the psychological frailty degree equal to or greater than a second threshold value and is determined to pose high risk, and the social frailty.

6. The information processing device according to claim 1, wherein
the processor compares the physical frailty degree with the psychological frailty degree, and determines which one of the physical frailty and the psychological frailty is at higher risk. and
the method for preventing the frailty is to reduce whichever of the physical frailty and the psychological frailty poses a higher risk, and reduce the social frailty.

7. The information processing device according to claim 3, wherein the psychological frailty estimation means estimates the psychological frailty degree of the subject by using a machine learning model which is optimized and trained to output the psychological frailty degree of the subject.

8. An information processing method performed by an information processing device including a detection device for detecting a state of a body of a subject, the information processing method comprising:
acquiring each facial image of a subject by an imaging device for capturing a body of the subject;
estimating a physical frailty degree of the subject based on each facial image;
estimating a psychological frailty degree of the subject based on the output of a detection device different from the imaging device; and
proposing a method for preventing frailty by displaying, on a user interface, a personalized recommendation of at least one dietary plan and exercise regime, based on the physical frailty degree and the psychological frailty degree of the subject.

9. A non-transitory computer-readable recording medium storing a program causing a computer including a detection device for detecting a state of a body of a subject to execute processing of:
acquiring each facial image of a subject by an imaging device for capturing a body of the subject;
estimating a physical frailty degree of the subject based on each facial image;
estimating a psychological frailty degree of the subject based on the output of a detection device different from the imaging device; and
proposing a method for preventing frailty by displaying, on a user interface, a personalized recommendation of at least one dietary plan and exercise regime, based on the physical frailty degree and the psychological frailty degree of the subject.
